(19) United States
(12) Patent Application Publication
(10) Pub. No.: US 2018/0360346 A1
(43) Pub. Date: Dec. 20, 2018

(54) ELECTRONIC INCENTIVE SPIROMETER

(71) Applicant: Yu-Fu Wu, Douliu City, (TW)

(72) Inventors: Yu-Fu Wu, Douliu City (TW); CHIH-SHEN LI, Hualien City (TW); CHUN-PENG HSU, New Taipei City (TW); YUAN-EN WANG, Taipei City (TW); Shabbir Syed Abdul, Taipei City (TW); Alon Dagan, Cambridge, MA (US)

(21) Appl. No.: 15/626,171

(22) Filed: Jun. 18, 2017

(51) Int. Cl. A61B 5/091; G01D 5/34; G01D 5/40

(52) U.S. Cl. CPC A61B 5/091; G01D 5/40; G01D 5/342

(57) ABSTRACT

An electronic incentive spirometer has a base and a sensing device. The base is provided with a breathing tube and at least one vertical tube. A floating ball is disposed in the vertical tube, one end of the vertical tube connected to the breathing tube. The sensing device is disposed on the vertical tube of the base, has a sensing unit corresponding with the vertical tube and a control circuit, and the sensing unit is electrically connected to the control circuit.

### BACKGROUND OF INVENTION

### 1. Field of Invention

The present invention relates to an electronic incentive spirometer, and more particularly to a reusable electronic incentive spirometer.

### 2. Description of Related Art

Traditional triflow incentive spirometer requires another person to record the number of breaths of patients, which might be incorrect due to temporary interruption. Also, traditional one-ball or three-ball incentive spirometer are simple mechanism devices, which is not able to record breathing training status. One conventional electronic incentive spirometer is designed for personal use, which can be expensive.

Therefore, it is desirable to provide an electronic incentive spirometer to mitigate and/or obviate the aforementioned problems.

### SUMMARY OF INVENTION

An objective of present invention is to provide an electronic incentive spirometer

In order to achieve the above mentioned objective, an electronic incentive spirometer has a base and a sensing device. The base is provided with a breathing tube and at least one vertical tube. A floating ball is disposed in the vertical tube, one end of the vertical tube connected to the breathing tube. The sensing device is disposed on the vertical tube of the base, has a sensing unit corresponding with the vertical tube and a control circuit, and the sensing unit is electrically connected to the control circuit.

Other objects, advantages, and novel features of invention will become more apparent from the following detailed description when taken in conjunction with the accompanying drawings.

### BRIEF DESCRIPTION OF DRAWINGS

FIG. 1 is a functional block drawing of an electronic incentive spirometer according to an embodiment of the present invention.

FIG. 2 is a schematic drawing of the electronic incentive spirometer according to the embodiment of the present invention.

FIG. 3 is a schematic drawing showing a wireless signal connection of the electronic incentive spirometer according to the embodiment of the present invention.

FIG. 4 is a schematic drawing showing a user using the electronic incentive spirometer according to the embodiment of the present invention.

FIG. 5 is a schematic drawing showing a user using the electronic incentive spirometer according to another embodiment of the present invention.

FIG. 6 is a front view of the embodiment of the present invention shown in FIG. 5.

FIG. 7 is a schematic drawing showing the electronic incentive spirometer connected to a single ball incentive spirometer according to the embodiment of the present invention.

FIG. 8 is another schematic drawing showing the electronic incentive spirometer connected to a single ball incentive spirometer according to the embodiment of the present invention.

### DETAILED DESCRIPTION OF PREFERRED EMBODIMENT

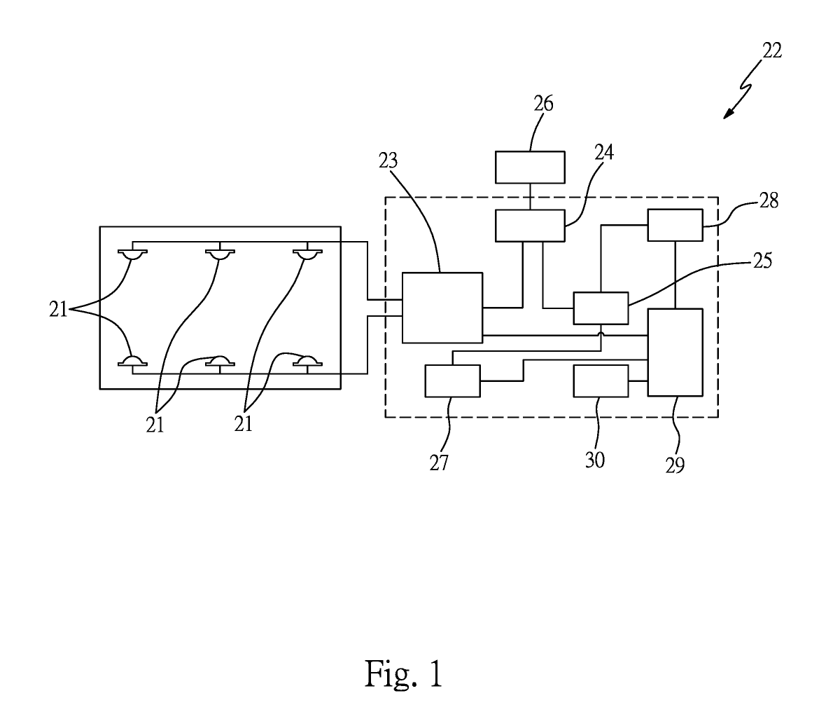

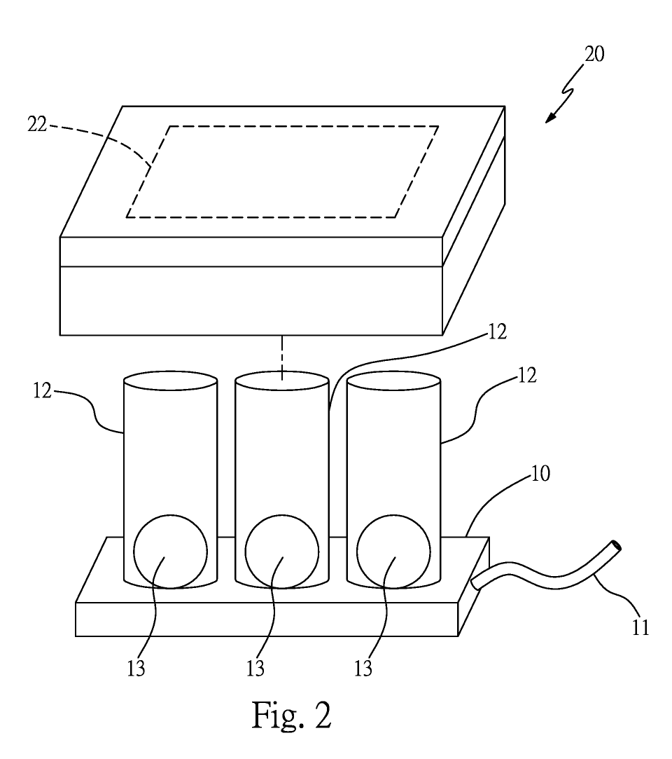

Please refer to FIG. 1 and FIG. 2. An electronic incentive spirometer comprises a base 10 and a sensing device 20. The base 10 is provided with a breathing tube 11 and three vertical tubes 12. A floating ball 13 is disposed in each vertical tube 12, one end of each vertical tube 12 is connected to the breathing tube 11, as shown in FIG. 2. The sensing device 20 is disposed on the three vertical tubes 12 of the base 10 and has a sensing unit 21 corresponding with each vertical tube 12 and a control circuit 22. The three sensing units 21 might all be a photointerrupter. The control circuit 22 has a GPIO 23, an IC 24, a micro-processor 25 and a counter 26, a RFID reader 27 and a wireless LAN module 28. The sensing units 21 are electrically connected to the GPIO 23 of the control circuit 22 and disposed on the upper portion of the vertical tubes 12. The IC 24 is electrically connected to the GPIO 23, the micro-processor 25 and the counter 26. , and the RFID reader 27 and the wireless LAN module 28 are electrically connected to the micro-processor 25. A power supply source 29 supplies the control circuit 22 with an AC/DC current and is controlled by a power switch 30.

As shown in FIG. 2, the sensing device 20 is attached onto the vertical tube 12, and each sensing units 21 are respectively corresponding to each vertical tube 12. After the application, the sensing device 20 can be detached from the incentive spirometer.

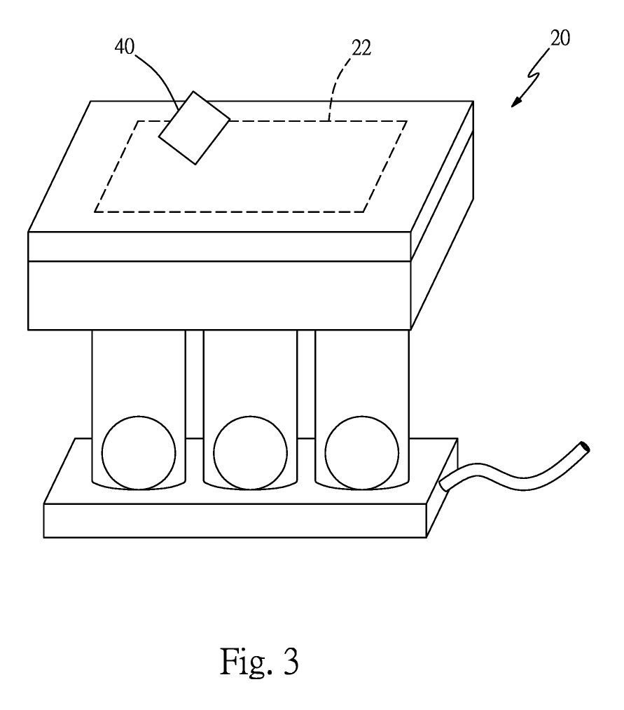

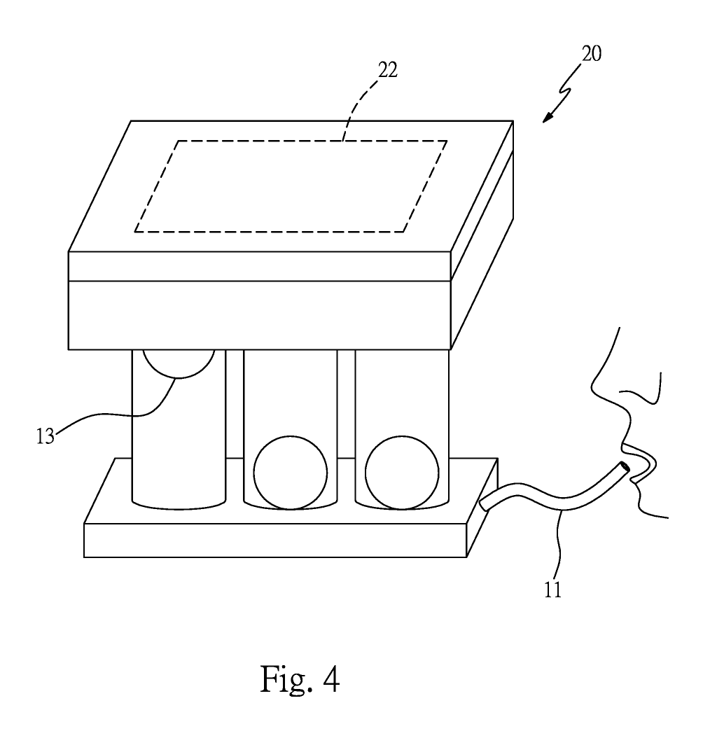

Please refer to FIG. 1 and FIG. 3 together, when the power switch 30 of the sensing device 20 is turned on, the control circuit 22 utilizes the RFID reader 27 to read an electronic Tag 40 belongs to a patient. As shown in FIG. 4, when the patient is exhaling to the breathing tube 11, the floating ball 13 floats upward and blocks the sensing unit 21. From one of the floating ball 13 floats up and blocks the sensing unit 21 till all three floating balls 13 float up to the sensing units 21, it is defined as one complete cycle. The GPIO 23 of the control circuit 22 sends signals to the IC 24 and the micro-processor 25 to provide data to the counter 26. Furthermore, the control circuit 22 utilizes the micro-processor 25 to send patient data obtained by the RFID reader 27 from patient's electronic tag 40 and breathing data to the wireless LAN module 28, such that medical staff can download the patient data.

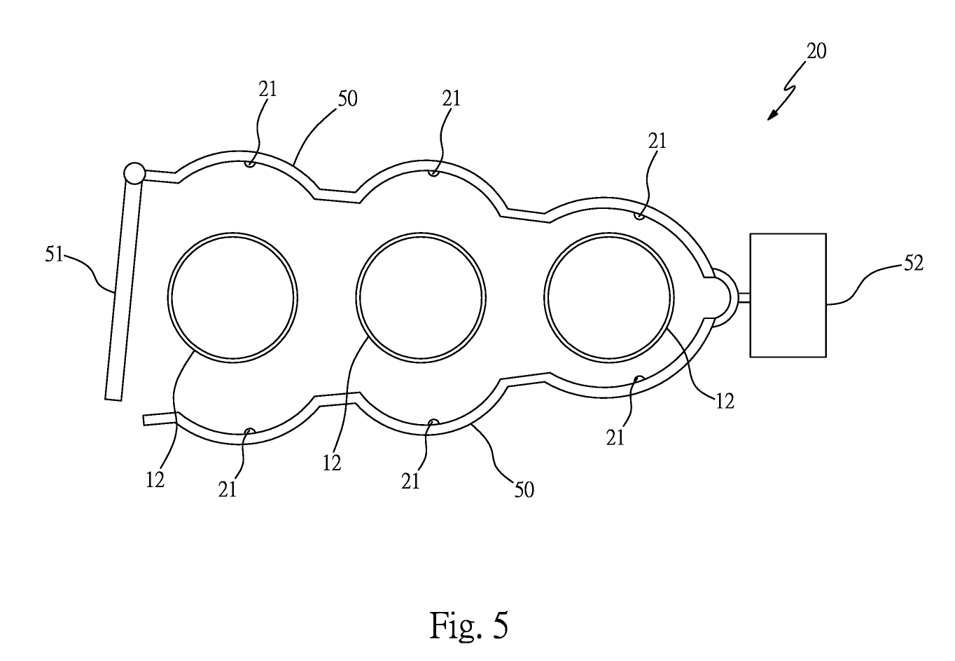

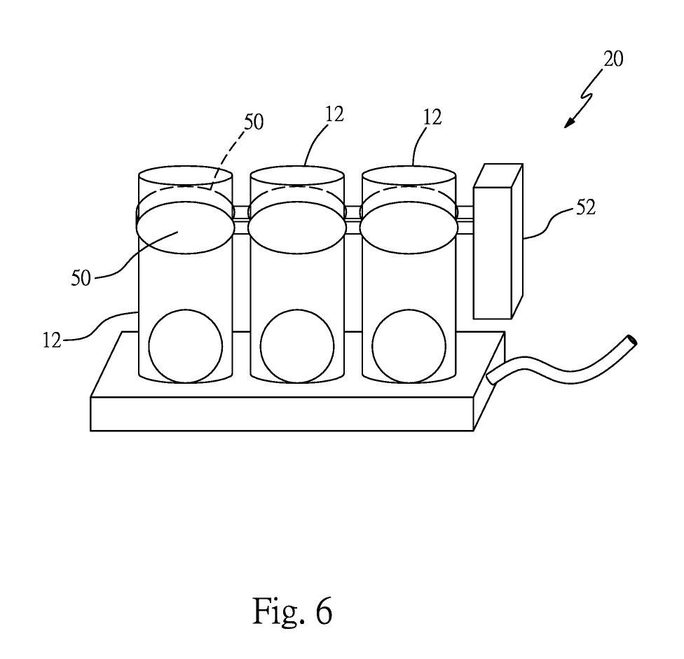

Please refer to FIG. 5 and FIG. 6. The sensing device 20 is attached onto a clip-on mechanism, the sensing unit 21 is separately disposed on two arms 50, and a fasten portion 51 secures the two arms 50 together around the vertical tube 12. The control circuit 22 is disposed in a box 52, and the arms 50 are pivoted with the box 52, such that various triflow incentive spirometer can be connected with the sensing device 20.

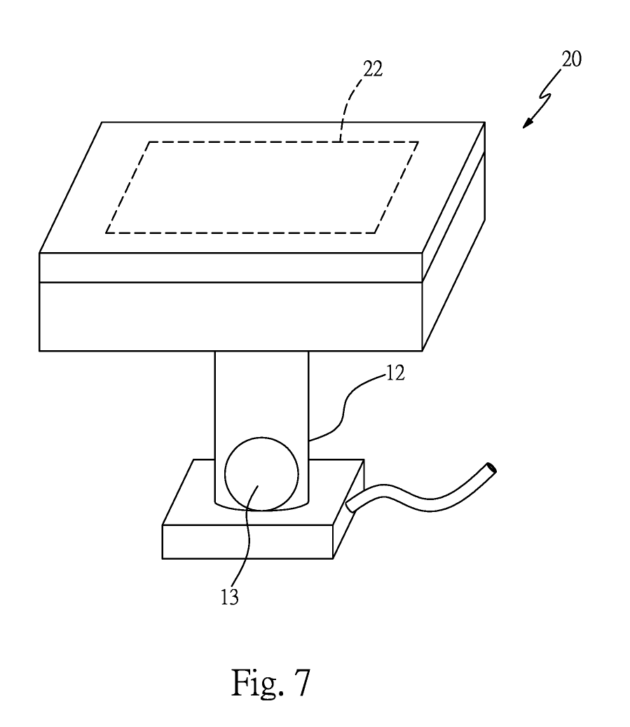

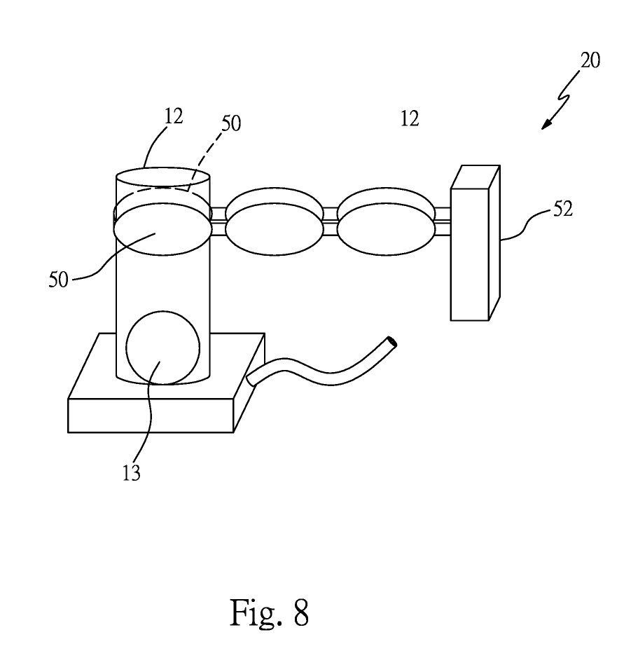

Please refer to FIG. 7 and FIG. 8. Another embodiment of the present invention can be applied on a single ball incentive spirometer, the sensing device 20 can be attached onto the incentive spirometer. The sensing unit may be an IR distance sensor, which is used for detecting the lift height of the floating ball to obtain a total breathing amount.

With the above-mentioned embodiments, the breathing data can be analyzed with related software for treatment determination.

Although the present invention has been explained in relation to its preferred embodiment, it is to be understood that many other possible modifications and variations can be made without departing from the spirit and scope of invention as hereinafter claimed.

What is claimed is:

1. An electronic incentive spirometer comprising:
a base provided with a breathing tube and at least one vertical tube; a floating ball disposed in the vertical tube, one end of the vertical tube connected to the breathing tube; and
a sensing device disposed on the vertical tube of the base, having a sensing unit corresponding with the vertical tube and a control circuit, and the sensing unit electrically connected to the control circuit.

2. The electronic incentive spirometer as claimed in claim 1, wherein the control circuit has a GPIO, an IC, a micro-processor and a counter.

3. The electronic incentive spirometer as claimed in claim 1, wherein the sensing unit may be a photointerrupter.

4. The electronic incentive spirometer as claimed in claim 1, wherein the sensing unit may be an IR distance interrupter.

5. The electronic incentive spirometer as claimed in claim 1, wherein the control circuit further comprises a RFID reader electrically connected to the micro-processor.

6. The electronic incentive spirometer as claimed in claim 1, wherein the control circuit further comprises a wireless LAN module electrically connected to the micro-processor.
